(19) United States
(12) Patent Application Publication
(10) Pub. No.: US 2007/0092398 A1
(43) Pub. Date: Apr. 26, 2007

(54) Pouch for sterilization of medical products

(76) Inventor: Duane Lyle McDonald, Neenah, WI (US)

Correspondence Address: KIMBERLY-CLARK WORLDWIDE, INC., 401 NORTH LAKE STREET, NEENAH, WI, 54956, US

(21) Appl. No.: 11/258,546

(22) Filed: Oct. 25, 2005

(51) Int. Cl. A61L 2/20

(52) U.S. Cl. 422/28; 206/364; 206/368; 206/370; 206/439; 422/292

(57) ABSTRACT

The present invention provides a gas permeable pouch which has various uses including as a sterilization pouch for items in need of sterilization. The pouch has a front panel and a back panel, each panel having a top edge margin, a bottom edge margin and two side edge margins wherein the top margin of the front panel is directly or indirectly joined to the top edge margin of the back panel and each side edge margin of the front panel is directly or indirectly joined to the one of the side edge margins of the back panel. A bottom panel having a perimeter with a perimeter edge margin. The bottom panel is directly or indirectly joined at the perimeter edge margin of the bottom panel to the bottom edge margins of the front side panel and back side panels with a line of juncture such that the bottom edge of the front panel and the bottom edge of the back panel are non-linear when the bottom panel is fully expanded. The front panel, back panel and bottom panel define a compartment capable of holding one or more articles and at least one the front panel, the back panel or the bottom panel at least partially has a portion containing a gas permeable material. This gas permeable material allows gas to pass through the material and contact the one or more articles contained within the compartment or allows gas to pass through the gas permeable material from the inside of the compartment to the outside of the container. Also disclosed is a method of sterilizing items used in a medical procedure using the gas permeable pouch of the present invention.

### FIELD OF THE INVENTION

The present invention relates generally to a gas permeable pouch which may be used in sterilization procedures, as well as other uses. In addition, the present invention relates to a method of sterilizing an item used in a medical procedure.

### BACKGROUND OF THE INVENTION

Sterilization of items used in medical procedures is vital to minimizing the spread of harmful and infectious agents to patients. Typically, the items used in medical procedures are placed into a sterilization container such as sterilization wraps, vented rigid containers or into breathable pouches. Breathable pouches are packaging for items used in medical procedures which generally have a gas permeable material joined to a gas impermeable material. All of these containers are generally gas permeable. These sterilization containers preserve sterility of the items contained therein, as well as the interior portion of these containers, after the containers and contents of the container have been through a sterilization procedure. The gas permeable material allows a gas sterilant to enter the breathable pouch, rigid container or wrap, thereby allowing the gas sterilant to contact the item to be sterilized in the pouch, container or wrap.

In gas sterilization procedures, the wraps, vented rigid containers or breathable pouches, along with an item to be sterilized that are placed within the wraps, vented rigid containers and breathable pouches are placed into a sterilization chamber. Examples of current gas sterilization procedures include, gas plasma sterilization, steam sterilization, ethylene oxide sterilization, hydrogen peroxide sterilization, and ozone sterilization. Other sterilization procedures, such as irradiation have also been used.

When gas sterilization procedures are used to sterilize items used in a medical procedure, it is necessary for the gas sterilant to penetrate sterilization container to contact the item used in a medical procedure. As a result, in the case of breathable pouches or wraps, a portion of the pouch or wrap must be gas permeable. Current sterilization pouches have been made with a variety of configurations. For example, the entire pouch material may be gas permeable or portions of the pouch, such as side seams, are made of a gas permeable material. In the case where the side seams are prepared from a gas permeable material, the front and back panels are typically prepared from a gas impermeable material. Sterilization pouches with one or more of the side seams being prepared from a gas permeable material are generally stored flat and are partially or completely opened to create a three-dimensional space capable of accommodating an item to be sterilize within the three-dimensional space. Once the item is placed within the pouch, the pouch is close or otherwise sealed. When these pouches and the items contained within each pouch are sterilized, the pouches frequently stacked on top of one another or are adjacent one another. It is possible that the side seams may become compressed or otherwise occluded during sterilization such that the gas impermeable materials of the front and back panels potentially come into contact with one another, resulting in the gas permeability of the pouch being greatly reduced, if not eliminated. When this occurs, there is a potential that the items to be sterilized may not be completely sterilized.

In addition, the current pouches with the gas permeable side seams are difficult to stack in a sterilization chamber. Often as the pouches are stacked, the stacks tend to be unstable and have the potential to fall over. This can result in extra time being needed to sterilize the items in need of sterilization, requiring careful stacking of the sterilization pouches, such that the pouches have the gas permeable materials of the pouches remaining unblocked by any of the impermeable materials used in the pouches, while keeping the stacks from falling over. This task can be time consuming and tedious.

There is a need in the art for a sterilization pouch which can be used to sterilize items used in medical procedures that can ensure proper sterilization and can easily be placed in a sterilization unit, without the problems of the prior pouches.

### SUMMARY OF THE INVENTION

Generally stated, the present invention provides a gas permeable pouch which has various uses including as a sterilization pouch for items in need of sterilization. The pouch has a front panel and a back panel, each panel having a top edge, a bottom edge, two side edges, a top margin at or near the top edge, a bottom margin at or near the bottom edge and two side margins at or near the two side edges wherein the top margin of the front panel is directly or indirectly joined to the top margin of the back panel and each side margin of the front panel is directly or indirectly joined to one of the side margins of the back panel. The pouch further has a bottom panel having a perimeter with a perimeter margin. This bottom panel is directly or indirectly joined at the perimeter margin to the bottom margins of the front side panel and back side panels using a line of juncture such that the bottom edge of the front panel and the bottom edge of the back panel are non-linear when the bottom panel is fully expanded. The front panel, back panel and bottom panel define a compartment capable of holding one or more articles. At least one of the front panel, the back panel or the bottom panel has a portion containing a gas permeable material. This gas permeable material allows gas to pass through the material and contact the one or more articles contained within the compartment or allows gas to pass through the gas permeable material from the inside of the compartment to the outside of the container.

In another embodiment of the present invention, the present invention provides a sterilization container for an item used in a medical procedure. The sterilization container allows an item used in a medical procedure to be sterilized prior to use in a medical procedure. The sterilization container has a front panel and a back panel, each panel having a top edge, a bottom edge, two side edges, a top margin at or near the top edge, a bottom margin at or near the bottom edge and two side margins at or near the two side edges wherein the top margin of the front panel is directly or indirectly joined to the top margin of the back panel and each side margin of the front panel is directly or indirectly joined to the one of the side margins of the back panel. The sterilization container further has a bottom panel having a perimeter with a perimeter margin. The bottom panel is directly or indirectly joined at the perimeter margin to the bottom margins of the front panel and back panel using a line of juncture such that the bottom edge of the front panel and the bottom edge of the back panel are non-linear when the bottom panel is fully expanded. The front panel, back panel and bottom panel define a compartment capable of holding one or more items used in a medical procedure. Contained within the compartment is an item used in a medical procedure which is in need of sterilization. At least one of the front panel, the back panel or the bottom panel has a portion containing a gas permeable material.

In another embodiment of the present invention, provided is a method for sterilizing an item used in a medical procedure. In this method, an item used in a medical procedure contained within a sterilization container is provided. The sterilization container has a front panel and a back panel, each panel having a top edge, a bottom edge, two side edges, a top margin at or near the top edge, a bottom margin at or near the bottom edge and two side margins at or near the two side edges wherein the top margin of the front panel is directly or indirectly joined to the top margin of the back panel and each side margin of the front panel is directly or indirectly joined to the one of the side margins of the back panel. The pouch further has a bottom panel having a perimeter with a perimeter margin. This bottom panel is directly or indirectly joined at the perimeter margin to the bottom margins of the front panel and back panels using a line of juncture such that the bottom edge of the front panel and the bottom edge of the back panel are non-linear when the bottom panel is fully expanded. The front panel, back panel and bottom panel define a compartment capable of holding one or more items used in a medical procedure. At least one of the front panel, the back panel or the bottom panel has a portion containing a gas permeable material. The sterilization container, with the item used in medical procedure contained within the compartment of the container, is exposed to a sterilization gas. In a further embodiment of this method of the invention, the sterilization container and medical item to be sterilized are placed into a sterilization chamber.

### BRIEF DESCRIPTION OF THE DRAWINGS

FIG. 1 shows a perspective view of the gas permeable pouch or container of the present invention.

FIG. 2 shows a cross-section side view of the pouch or container of the present invention.

FIG. 3 shows a perspective view of a gas permeable pouch or container viewing slightly below a plane of the bottom edge of the pouch.

FIGS. 4A, 4B and 4C each show a front view of the pouch or container of the present invention with an opening device.

FIG. 5 shows a front view of the pouch or container of the present invention with a stay to maintain the bottom of the pouch in an arced and opened position.

FIG. 6 shows a cross-section view of the pouch or container of FIG. 5 with the location of the stay.

FIG. 7 shows a perspective bottom view of a pouch of the present invention.

FIG. 8 shows a cross section view of a stay useable in the present invention.

FIG. 9 shows a possible stacking configuration of the pouches or containers of the present invention.

FIG. 10 shows a different stacking configuration for the pouches or containers of the present invention.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As used herein, the term “self-supporting” means that the pouch has the property to stand upright and support the weight of the pouch. In addition, as used herein, self-supporting is also intended to indicate that the pouch of the present invention may also support the weight of the pouch and any items contained within the compartment of the pouch.

As used herein, the term “sterilization” refers to a wide variety of techniques employed to attenuate, kill or eliminate harmful or infectious agents. Examples of sterilization procedures include, for example, gas plasma sterilization, steam sterilization, ozone sterilization, hydrogen peroxide sterilization, ethylene oxide sterilization and irradiation.

The term “sterilizing conditions” refers to a combination of a concentration of sterilant and a time exposure interval which will sterilize an object which is subjected to a sterilant within a sterilizing container. Sterilizing conditions may be provided by a wide range of sterilant concentrations in combination with various time intervals. In general, the higher the concentration of a sterilant, the shorter a corresponding time interval is needed to establish sterilizing conditions. Accordingly, the effective amount of a sterilant may vary depending upon the length of exposure of the medical supplies to the sterilant.

As used herein, the term “gas permeable” is intended to mean a material which will allow gas to pass through the material but fails to allow airborne microbes, bacteria, viruses and mixtures thereof to pass through the material. Gas permeable materials are also referred to in the art as breathable materials.

As used herein, “gas impermeable” is intended to mean a material which does not readily allow gas to pass through the material. In addition, the gas impermeable material also fails to allow airborne microbes, bacteria, viruses and mixtures thereof to pass through the material.

As used herein, the term “opening device” refers to a mechanism which facilitates the opening of the pouch or container by a user.

As used herein, the term “closing device” refers to a mechanism which facilitates closing of the pouch or container by the user.

### DETAILED DESCRIPTION OF THE INVENTION

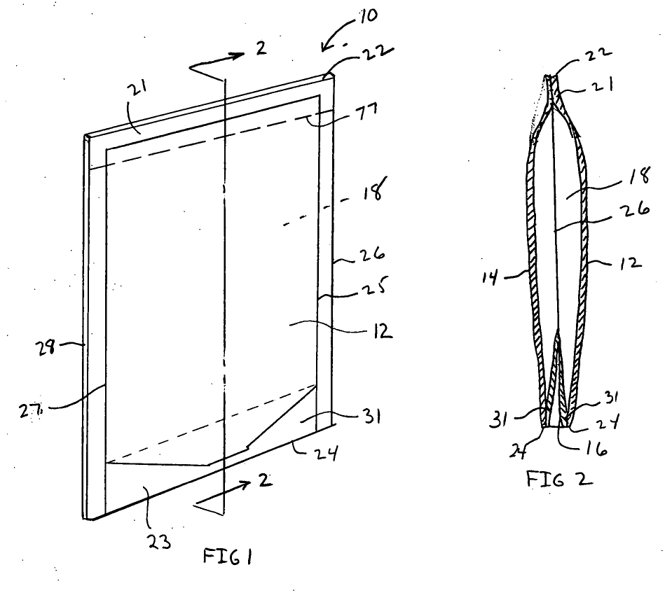

In order to obtain a better understanding of the present invention, attention is directed to FIGS. 1-8. The pouch 10 or container 10 has a front panel 12 and a back panel 14, each panel having a top edge 22, a bottom edge 24, two side edges 26, 28, a top margin 21 at or near the top edge 22, a bottom margin 23 at or near the bottom edge 24, and two side margins 25, 27 at or near the two side edges 26, 28, respectively. Collectively, the front panel and the back panel may also be referred to herein as the “side panels”. The front panel 12 is directly or indirectly joined or connected to the back panel 14 along the top margin 21, and at least partially along the side margins 25 and 27. The pouch has a bottom panel 16 having a perimeter 32 with a perimeter margin 31. This bottom panel 31 is directly or indirectly joined at the perimeter margin 31 to the bottom margins 24 of the front panel 12 and back panels 14. The front panel 12, back panel 14 and bottom panel 16 define a compartment 18 capable of holding one or more articles. At least one of the front panel 12, the back panel 14 or the bottom panel 16 has a portion containing a gas permeable material. This gas permeable material allows gas to pass through the material and contact the one or more articles contained within the compartment and/or allows gas to pass through the gas permeable material from the inside of the compartment to the outside of the pouch.

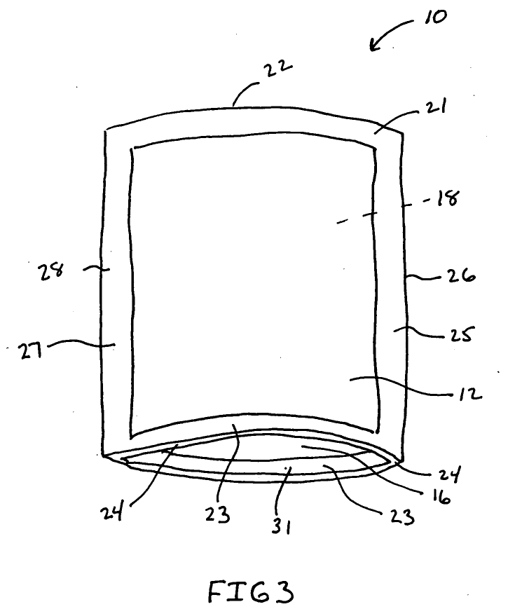

The pouch can have a flat configuration as shown in FIG. 1 where the bottom panel 16 is in the form of a gusset, as shown in FIG. 2. The bottom panel 16 can be flattened by pulling the side panels near the bottom edge outward or by exerting a force from the inside of the compartment area of the pouch. Due to the shape of the bottom panel 16 and the manner in which the bottom panel is joined to the, the front and back side panels 12 and 14 tend to bow outward forming an arc. Stated another way, the bottom panel is joined with the front and back panels with a line of junction that causes the front and back panel to be non-linear when the bottom panel is fully extended. This extends the area which the pouch covers, thereby making the pouch self-supporting. This is shown in FIG. 3.

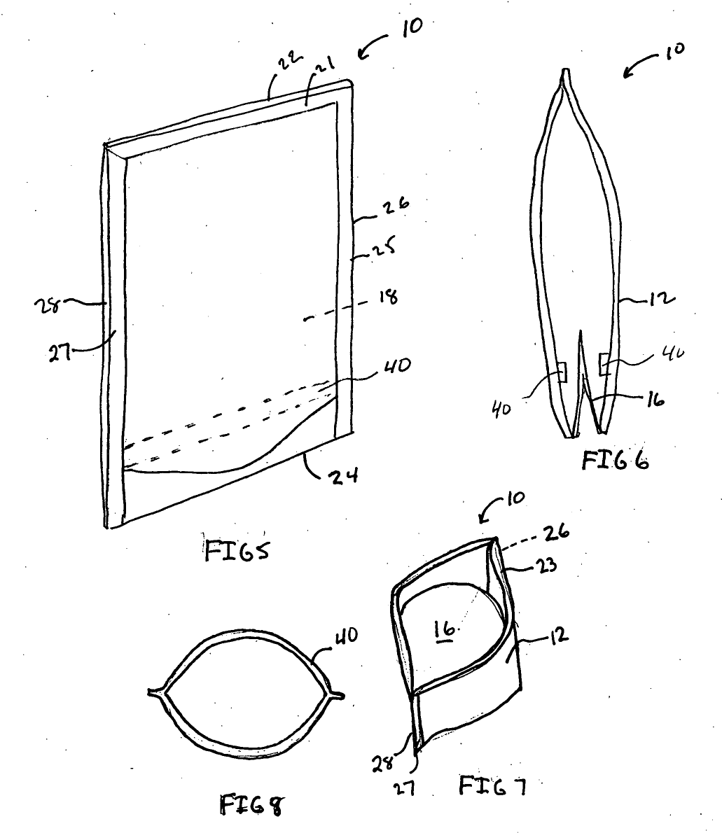

In the configuration of the pouch 10, the side panels at or near the bottom edge 24 have a natural tendency to extend outwards and away from the contents within the compartment 18. This is shown in FIG. 3 where the bottom edge 24 has a perimeter 31 having a generally oval or elliptical shape. By having the side panels of the pouch arc out as shown in FIG. 3 and further shown in FIG. 7, the pouch has the ability to support its own weight and be free-standing. This can result in an advantage over the prior breathable packages since the pouch of the present invention can stand in an upright position. As a result, the pouches of the present invention do not need to be stacked on one another when several pouches and the contents of the pouches are sterilized using gas sterilization.

To further assist in the self-supporting nature of the pouches of the present invention, additional embodiments provide reinforcement to the side panels so that they remain in an arc configuration. One method to provide reinforcement is to use a sealing method between the side panels which provides strength to the structure of the pouch. For example, heat sealing and adhesive bonding between the front and back panels and the front and back panels and the bottom panel can result in greater rigidity in the pouch structure by selecting particular heat sealing patterns or adhesive patterns over others. As an example, using a wider heat seal area or a wider adhesive pattern will impart more strength and rigidity than a narrower heat seal area or adhesive pattern. In addition, selection of the adhesive used or the amount of the adhesive used to join the side panels to each other or to the bottom panel can also strengthen the side panels and bottom panel so that the side panels will remain in an arced configuration.

Other methods of reinforcing the pouch so that it maintains the arced configuration include adding additional elements to the pouch. For example, a heat sensitive material could be placed on the bottom panel or on the side panels that will set when exposed to heat or irradiation. When the heat sensitive material is set, it will lock the bottom portion of the side panels or the bottom panel in place such that the lower portion of the front and back side panels are set in the arced configuration. In gas sterilization processes, heat is often used in conjunction with the sterilization gas. This locking of the front and back panels and/or bottom panel will stiffen the pouch a sufficient amount so that the pouch will stay in an upright position during the gas sterilization process.

Other methods of ensuring that the pouch will stay in the arced configuration during the sterilization gas, ensuring that the sterilant will contact the items in the compartment, include providing a bottom panel with sufficient stiffness that will hold the side panels in an arced configuration. Stiffness of the bottom panel can be imparted by a selection of stiffer material for the bottom panel. One method to provide greater stiffness is to select a material for the bottom panel that has a higher basis weight or thickness. In addition, portions of the bottom panel may be treated with a composition which will increase the stiffness in all parts of the bottom panel. Other methods of increasing stiffness of the bottom panel are to provide the bottom panel with pleats. If the bottom panel is provided with accordion pleats, these accordion pleats will tend to resist compression caused by the side panels and the weight of the items in the pouch. Additional elements may also be placed on the bottom panel including materials that will lock in place when taken out of a folded configuration, such as shape memory elements.

Other methods of ensuring that the pouch will stay in the arced configuration are to provide stays on the side panels or the bottom panel. Referring to FIG. 5, an extensible stay 40 (shown in phantom in FIG. 5) may be provided to ensure that the bottom edge of each side panel remains separated and the bottom panel is exposed when gas permeable pouch is subjected to a sterilization gas. The stay 40 may be located on the inside of the pouch, as is shown in FIG. 5, between pouch layers if more than one layer is present in the pouch side panels or may be located on the outer surface of the pouch. Generally, the stay is desirably out of view and only present on the inside of the pouch, as is shown in FIG. 5. The stay 40 may be located anywhere on the inside surface of the pouch but is generally located near the bottom edge 24 or the bottom panel of the pouch 10. As shown in FIG. 5, the stay 40 is attached to the inner surface of sidewalls 12 and 14 by using, for example, heat and pressure, an adhesive or mechanical fastening means or other means to keep the stay 40 in place. Stay 40 is made of a relatively stiff, resilient material having a memory. The term “memory” is intended to mean the phenomenon where a material returns to its original, unstressed configuration after having been deformed to a stressed configuration and the deforming force has been removed. When extensible stay 40 is secured inside sealed pouch 10 as shown in FIGS. 1 and 2, top seal 21, sidewalls 12 and 14, and the upper portions of side seals 18 and 19 cooperate in holding extensible stay 40 in its collapsed, stressed configuration.

In the present invention, suitable materials which may be used as the gas impermeable material include, for example, but are not limited to, polymeric plastic films, foils, paper, paper composites, fibrous webs and the like, laminates of one or more of these materials or a combination thereof of these materials. In addition other materials which are not gas permeable may also be used. The gas impermeable material may be a single layer or a laminate of two or more layers.

Suitable materials can be made from polymeric materials such as polyethylene, polypropylene, polyester, nylon, and the like, as well as any combination thereof. Plastic film materials include, for example, a low density polyethylene (LDPE) film, a LDPE/LLDPE (linear low density polyethylene) film laminate, a LDPE/MDPE (medium density polyethylene) film laminate, a LDPE/HDPE (high density polyethylene) film laminate, a ethylene-vinyl alcohol (EVOH) or the like. In addition, films made from a polyethylene/polypropylene combination may also be used. Films coated with metal coatings, also known as foils may also be used. In one embodiment of the present invention, the film materials used in the present invention include a polyolefin film, such as a polyethylene or polypropylene film. The thickness of the film can essentially be any thickness, provided that the film has sufficient strength that the articles contained within the compartment of the pouch do not puncher or otherwise compromise the film or the pouch.

It is also possible that the gas impermeable material is a laminate of a gas impermeable material and a gas permeable material. Examples of such laminates include, nonwoven/film laminates. These laminates may be beneficial to obtain a cloth-like feel to the outer or inner surface of the pouch or to reinforce or protect the film material from damage caused by the articles in the compartment of the pouch or from elements outside of the pouch.

Essentially any gas permeable material may be used in the present invention, provided that the material is permeable to a sterilizing gas but impermeable to airborne microbes, bacteria, viruses and mixtures thereof. Suitable gas permeable materials useable in the present invention include, for example, medical grade paper, nonwoven materials and other similar gas permeable materials. Generally, gas permeable materials which may be used in the present invention are permeable to water vapor and have a minimum water vapor transmission rate (WVTR) of about 300 g/m² /24 hours, calculated in accordance with ASTM Standard E96-80. Suitable medical grade paper includes, for example, AMCOR PLP reinforced coated paper available from AMCOR, Limited.

Suitable nonwoven materials useable as the gas permeable material of the pouch of the present invention include, for example, airlaid nonwoven webs, spunbond nonwoven webs, meltblown nonwoven webs, bonded-carded-webs, hydroentangled nonwoven webs, spunlace webs and the like. The method of manufacturing each of these materials is known in the art. Laminates of these materials may also be used.

Of these nonwoven materials, the fibrous material web may comprise a nonwoven meltblown web. Meltblown fibers are formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al. Generally speaking, meltblown fibers may be microfibers that may be continuous or discontinuous, and are generally smaller than 10 microns in diameter, and are generally tacky when deposited onto a collecting surface.

The nonwoven material web may be a nonwoven spunbond web. Spunbonded fibers are small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel, et al., U.S. Pat. No. 3,692,618 to Dorschner, et al., U.S. Pat. No. 3,802,817 to Matsuki et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al. Spunbond fibers are generally not tacky when they are deposited onto a collecting surface. Spunbond fibers can sometimes have diameters less than about 40 microns, and are often between about 5 to about 20 microns.

The nonwoven material web may also comprise a laminate material such as a spunbond/meltblown/spunbond, or SMS, material. A typical SMS material is described in U.S. Pat. No. 4,041,203 to Brock et al. Other SMS products and processes are described, for example, in U.S. Pat. No. 5,464,688 to Timmons et al.; U.S. Pat. No. 5,169,706 to Collier et al.; and U.S. Pat. No. 4,766,029 to Brock et al. Generally, an SMS material will consist of a meltblown web sandwiched between two exterior spunbond webs. Such SMS laminates have been available commercially for years from Kimberly-Clark Corporation under marks such as Kimguard®). The spunbonded layers on the SMS laminates provide durability and the internal meltblown layer provides porosity.

In another embodiment of the present invention, the permeable material may be a laminate of a film layer and a fibrous material layer which have been laminated together by any lamination technique known to those skilled in the art. Examples of these laminates include, for example, spunbond-film laminates (SF), and other such laminates. Again, it is necessary for the laminate to be gas permeable. In one embodiment, the material for the wrapper component are prepared from a film/spunbond laminate material available from Kimberly-Clark Corp, and known as HBSTL (“highly breathable stretch thermal laminate”), and which material is further disclosed in U.S. Pat. No. 6,276,032, the entire disclosure of which is hereby incorporated herein by reference.

Suitable lamination means which may be used to form the gas permeable laminate materials include, but are not limited to, adhesives, needle punching, ultrasonic bonding and thermomechanical bonding as through the use of heated calendering rolls. Such calendering rolls will often include a patterned roll and a smooth anvil roll, though both rolls may be patterned or smooth and one, both or none of the rolls may be heated. Calendering may also be used to place an aesthetic pattern defined in the laminated material.

Other materials for the gas permeable material and the gas impermeable materials may be used for their properties. For example, if it is necessary for the compartments to be expandable, elastic materials, including elastic webs and elastic nonwovens may also be used. Again, the only requirement is that the one of the panels is gas permeable.

Each of the front and back panels and the bottom panel may have topical treatments applied thereto for more specialized functions. Such topical treatments and their methods of application are known in the art and include, for example, alcohol repellency treatments, anti-static treatments, non-slip treatments and the like, applied by spraying, printing, dipping, or other methods known to those skilled in the art. An example of such a topical treatment is the application of ZELEC® antistatic neutralized mixed alkyl phosphates (available from E.l. DuPont, Wilmington, Del.). Non-slip treatments are placed on the outside of the pouch which aid a user to grab the pouch and open it. Often users must open the pouches wearing protective articles, such as gloves, when the pouch compartment contains items used in a medical procedure. Having gloves donned may make it difficult to open the pouch to retrieve the items contained within the compartment. Non-slip treatments usable in the present invention can be formed in a variety of manners including applying a coating to the entire surface of the pouch or applying a coating in discrete areas on the pouch. The coating should have a higher coefficient of friction than the material used to prepare the pouch. Examples of such non-slip treatments include, but are not limited to, placing an elastomeric material on at least a portion of the surface of one or more side panels or the bottom panel.

In another embodiment of the present invention, the gas permeable material may be prepared from a material which can be electret treated, such as from a polyolefin containing materials. Electret treating the gas permeable material may further enhance the ability of the gas permeable material to prevent airborne microbes, bacteria, viruses and the like from penetrating the gas permeable material. Airborne microbes, bacteria, viruses may be attracted to the gas permeable material due to the electret treatment. Electret treating materials is known in the art and is described in, for example, U.S. Pat. No. 5,401,446. Electret treatment involves subjecting the material to a pair of electrical fields having opposite polarities. Each electrical field forms a corona discharge which is imparted to the material. Other means of electret treating materials are well-known and include process, such as, thermal, liquid contact and electron beam methods.

The panels of the pouch of the present invention may be directly or indirectly connected to one another. When they are directly connected together, techniques known to those skilled in the art, including, but not limited to, heat sealing, stitching, and adhesive sealing. Any method know to those skilled in the art may be used, provided that the panels are sealed together such that the seals are impermeable to airborne microbes, bacteria, viruses and mixtures thereof. Ideally, the sealing or joining should create an air-tight seal. Alternatively, the panels may be joined together with an intervening material. For example, it is possible to place a gas permeable material between the front and back panels and join this gas permeable material to the front and back panels using the same joining techniques described above. This may be advantageous when the front and back panels are each a gas impermeable material and the bottom panel is a gas permeable material. In another embodiment of the present invention, an intervening material may be placed between the front and back panels to facilitate expansion of the pouch when needed. The intervening material may form a gusset to allow the pouch to be expanded as necessary when the pouch is filled. In the alternative, the gas impermeable material may used to join the front and back panels together.

In one embodiment of the present invention, the front and back panels are prepared from a material which is gas impermeable and the bottom panel is prepared from a gas permeable material. In one embodiment of the present invention, the front and back panels are prepared from a polymeric film, such as a polyethylene film and the bottom panel is prepared from a breathable material, such as a nonwoven web, or a medical grade paper. One particular nonwoven web is a spunbond-meltblown-spunbond nonwoven web.

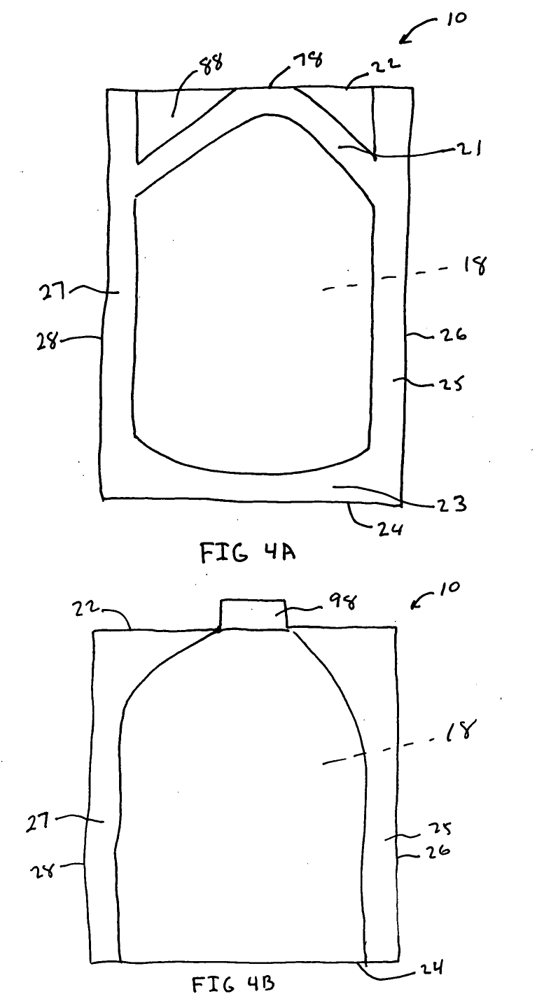

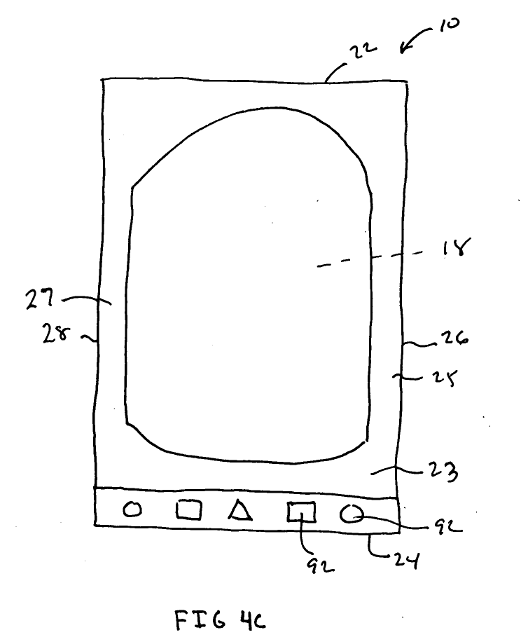

The pouches of the present invention may also be provided with opening devices. As is shown in FIGS. 1, 4A, 4B and 4C, an opening device that facilitates access to the items stored within the compartment may also be provided. The opening device shown in FIG. 1 is a line of weakness 77 provided one the front and back panels somewhere below the top margin 21. Due to the bonding at the side edges 25 and 27, which strengthens the pouch material in these areas, it is desirable that the line of weakness 77 extend through the side margins 27 and 25 so that the bonded side margins can be easily torn through when opening of the pouch is desired. To open the pouch with the line of weakness, a user will typically start near one of the side edges 25 or 27 and tear the pouch along the line of weakness. In addition, a notch (not shown) or other starting point for the opening may also be provided on the pouch. When the line of weakness is torn by the user or person opening the pouch, access to the compartment and the items within the compartment is provided. Although the line of weakness is shown as a line parallel with the top edge 22, it is noted that the line of weakness may be at an angle to the top edge. In addition, the line of weakness does not have to extend from one side edge to the other side edge, but may stop short of one of the side edges. Generally, it is desirable from an easy of opening standpoint that the line of weakness extends to at least one of the side edges.

Other opening devices types may also be used in the present invention. One popular opening device for breather pouches is a chevron seal, which is shown in FIG. 4A. In the chevron seal, the top margin 21 is not parallel with the top edge 22. In a chevron seal 78, areas 88 are provided to the user or opener of the pouch to grasp the side panels near the top edge 22 with the user's fingers. By giving the user a place to grasp the pouch material, the chevron seal is relatively easy to open, even when the person opening the pouch is wearing protective equipment, such as gloves. A further opening device is shown in FIG. 4B opening devices. In FIG. 4B the pouch is provided with tabs 98 on the front and back panels for a user or person opening the pouch a place to grasp the pouch 10 for opening. By having the chevron seal or the sealing configuration shown in FIG. 4B, the pouch is open in a manner similar to opening of a banana, such that the hand/arm of the opener does not come in contact with the sterile items. In addition, the non-sterile sides (outside surface) of the pouch tends not to come into contact with the sterile items in the pouch, thereby reducing the chance of accidental contamination of the items which have been sterilized during the open of the pouch of the present invention.

In another embodiment of the present invention, margins where the front and back panels are joined together may peelable to further facilitate opening of the pouch. For the margins to be peelable, the margins when subjected to a suitable tearing stress, are readily separated from one another. Conventional peelable seals or seams are well known to those skilled in the art and the methods of achieving pealable seals or seams are also well known. For example, before joining the front and back panels together, the margins to be sealed are provided with a release coating that will reduce seam strength. Many conventional release coatings may be provided to the margin areas. As a result, a person can use less force to open the pouch or break the seam or seal with less force than would be required to peal the seam or seal apart if the release coating was not applied. Other method of creating a release seal include selecting adhesives which will tend to release when a tearing stress is applied. Examples of such adhesives include pressure sensitive adhesives. It is desirable that the seals or seams in the opening structures shown in FIGS. 4A and 4B can be opened or broken without the use of excessive force. If too much force is required to open the seam or seal, it may be difficult to open the pouch or the pouch will tend to open rapidly due to the force needed, resulting in the items in the compartment being ejected from the pouch during opening.

In another embodiment of the present invention, the pouch may be provided with a closing device. Any closing device may be used, provided the closing device can seal the pouch and prevent airborne microbes, bacteria, viruses and mixtures thereof from entering the sealed pouch. Examples of closing devices include tongue and groove sealing devices, adhesives and the like. When an adhesive closing device is used, the adhesive may be protected with a peel strip which is removed before the pouch is sealed. The closing device of the present invention may also double as the opening device.

In a further embodiment of the present invention, the pouch can yet have some additional features. As is shown in FIG. 4C, the bottom margin may be a distance y from the bottom edge. By providing the extra distance y, the person opening the pouch may be able to grasp the contents of the container with one hand through the bottom panel and open the side margin seals and the top margin seal, by pulling the top edge towards the bottom edge. By being able to grasp the item in the pouch, the item is stabilized during the opening procedure which could prevent the item from being ejected from the pouch during opening. The bottom panel could be configured to allow the person opening the pouch to grasp the items in the compartment by providing, for example, the bottom panel has two spaced apart vertically extending folds (i.e. following the side walls), one fold to accommodate a thumb, the other to accommodate fingers to form a mitt-like structure to hold the contents.

In addition, often the sterilized items are usually transferred to a sterile surface by a sterile person. The contents of a sterilized package are seldom left in or on the opened packaging outsiders who typically open the pouches are not sterile. That is, typically the package or pouch containing the sterilized items often opened by a ‘non-sterile’ person (e.g. circulating nurse) and handed (untouched) to a ‘sterile’ person (e.g. surgeon, attending staff). By having the extra length between the bottom edge and the bottom margin, there is less chance that a non sterile person opening the package would likely touch the sterile items since one of their hands is under the pouch side panels near the bottom panel.

Also shown in FIG. 4C, the legs or arched portions of the side panels may be provided with cutouts 92 below the bottom margins on the side panels. The cutouts ensure adequate ingress/egress of sterilant gas to the bottom panel. The size and shape of the cutouts should be selected so as not to compromise the self-supporting feature of the pouch. As a result, the pouch may be set on a solid surface in a sterilization chamber and still have the contents of the pouch sterilized in a timely and efficient manner. In addition, the cut-outs may decrease the time to sterilize the contents of the compartment.

Other features which may be provided to the pouch of the present invention include, providing the pouch with a pre-printed or printable surface. The pre-printed surface would provide information or instructions about opening the package or the contents of the package. The printable surface would be useful for marking with a permanent marker to identify contents of the package or other notes, such as the time and date the item within the pouch was sterilized.

To sterilize the pouch and the items contained within the pouch, the pouch and items contained therein are exposed to a sterilization gas. Typical gas sterilization procedures include, for example, gas plasma sterilization, steam sterilization, ethylene oxide sterilization, hydrogen peroxide sterilization, and ozone sterilization. Typically, the pouch with the items contained therein is placed into a sterilization chamber. In the present invention, suitable sterilization chambers include those which can be used for gas sterilization. While the pouches of the present invention are designed for gas sterilization, this does not mean that other sterilization procedures, such as irradiation, cannot be used with the pouch. It is believed that the pouch of the present invention can be used in most sterilization procedures, provided that the materials in which the pouch is prepared are stable in the sterilization procedure used.

The pouch of the present invention may be placed in the sterilization chamber in an upright position with the top edge of the sterilization container being the uppermost portion of the sterilization container. With the arced side panels acting as legs the pouch is able to support its own weight and stand upright in the sterilization chamber. In an embodiment of the present invention, the pouch of the present invention is able to support its own weight and the weight of the item in the pouch. Stated another way, the pouch is self-supporting with the item contained within the compartment.

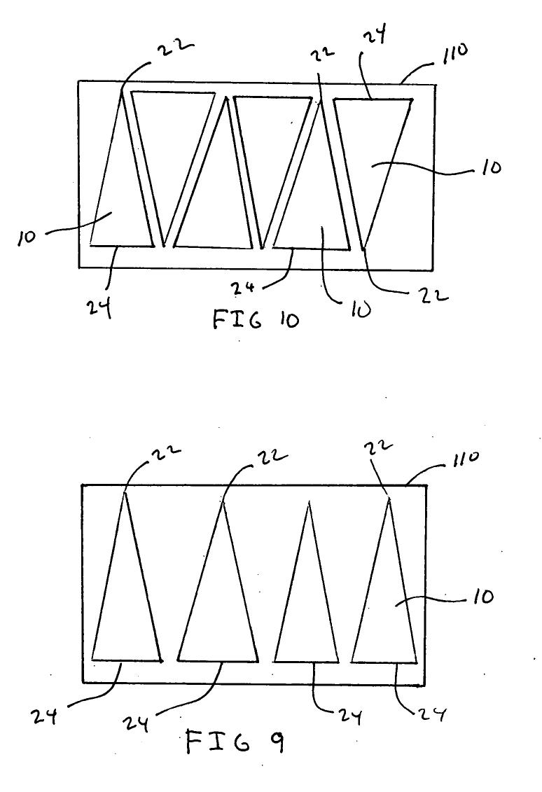

When placing the pouch with the item to be sterilized in the sterilization chamber, the user should check to make sure the base of the front and back side panels are arced outward to ensure that the sterilization gas will be able to pass through the gas permeable material of the bottom panel, if the bottom panel is the gas permeable material of the pouch. The pouches may be placed in any order into the chamber provided the gas permeable portion is readily accessible for the gas sterilant. The pouches may be placed in the chamber 110 in an upright configuration, as is shown in FIG. 9. That is, the top edge 22 of each pouch 10 is the uppermost portion of the pouch, unless one or more tabs 98 are present. In addition, if the pouches are dimensionally stable, it is possible to stack the pouches in other configurations. For example, the sterilization containers are placed in the sterilization chamber wherein the orientation of the containers (pouches) are alternated such that some of the sterilization containers have the top edge 22 as the upper most portion of the sterilization container and one or more of the sterilization containers 10 are placed in the sterilization chamber in an upside-down position, such that the top edge 22 of the sterilization container is the lowermost portion of the sterilization container and the bottom edge 24 is the uppermost portion of the container. This is shown in FIG. 10. Other possible configurations may also be used, including laying some or all of the sterilization containers in the sterilization chambers on their side seams.

Once the sterilization containers or pouches of the present invention, containing the items to be sterilized are placed in the sterilization chamber, containing the sterilization chamber is closed, and a gas sterilant is introduced into the container. The amount of time the items in the compartment are subjected to the gas sterilant depends on various factors, including the type of gas sterilant used, the number of sterilization container placed in the the sterilization container as well as other factors. Those skilled in the art will be able to determine the appropriate amount of time the gas sterilant should remain in the chamber based on these and other factors.

Once sterilized, the sterilization containers are removed from the chamber and the container or pouch with the sterilized items contained therein are stored or placed for use. The sterilization containers of the present invention have some advantages, some of which are described above. One big advantage is the items in the chamber can be dispensed from the container when the container is in an upright position. By having the sterilization container able to dispense the sterilized item from an upright position, the amount of sterile surface space needed in the pre-operating room for dispensing the items can be greatly reduced. That is, sterilized items are usually transferred to a sterile surface. The contents or items in a sterilized package are seldom left in the opened pouch or left on the outside of the opened packaging, since the outsides are not sterile. Typically, the package is opened by a ‘non-sterile’ person (e.g. circulating nurse) and handed (untouched) to a ‘sterile’ person (e.g. surgeon, attending staff). The opening feature of the present invention further provides this upright container dispensing, in conjunction with the self supporting feature of the pouch or sterilization container of the present invention.

In another embodiment of the present invention, the item to be sterilized is first placed into the compartment of the pouch or container and the container is closed or sealed. This is typically by closing the closing device on the pouch or container.

Typical items which may be sterilized in the pouch of the present invention include items that are used in medical procedures which need sterilization. Examples of such items include, for example, a protective garment, a protective covering, a wound covering, a suture, a clamp, a scalpel, a retractor, forceps, scissors, a blade handle, a glove, a needle, a sponge, a syringe, a receptacle, a sealed vessel holding a therapeutic agent or a combination thereof, in addition other items used in medical procedures not specifically mentioned. Each pouch may contain one or several of items in the compartment of the pouch.

The pouch of the present invention could also be used as a container for other various items, which will readily apparent to those skilled in the art.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A gas permeable pouch comprising:
a front panel and a back panel, each panel comprises a top edge, a bottom edge and two side edges, a top margin at or near the top edge, a bottom margin at or near the bottom edge and two side margins at or near the two side edges, wherein top margin of the front panel is directly or indirectly joined to the top margin of the back panel, each side margin of the front panel is directly or indirectly joined to the one of the side margins of the back panel;
a bottom panel comprising a perimeter having a perimeter margin located at or near the perimeter, wherein the perimeter margin of the bottom panel is directly or indirectly joined to the bottom margin of the front panel and the bottom margin of the back panel and the bottom panel is joined with the bottom margin of the front panel and the bottom margin of the back panel with a line of juncture such that the bottom edge of the front panel and the bottom edge of the back panel are non-linear when the bottom panel is fully expanded, and
wherein the front panel, back panel and bottom panel define a compartment capable of holding one or more articles and at least one the front panel, the back panel or the bottom panel at least partially comprises a gas permeable material.

2. The gas permeable pouch of claim 1, wherein the bottom edge of the front panel and the bottom edge of the back panel form a bottom perimeter of the pouch and the bottom perimeter has a generally elliptical shape when the bottom panel is fully extended.

3. The gas permeable pouch of claim 1, wherein the gas permeable material is impermeable to airborne microbes, bacteria, viruses and mixtures thereof.

4. The gas permeable pouch of claim 3, wherein the gas permeable material comprises a fibrous material.

5. The gas permeable pouch of claim 4, wherein the gas permeable material comprises a nonwoven material or paper material.

6. The gas permeable pouch of claim 1, wherein the bottom panel comprises the gas permeable material.

7. The gas permeable pouch of claim 6, wherein the front panel and the back panel comprise a gas impermeable material.

8. The gas permeable pouch of claim 7, wherein the gas impermeable material comprises a member selected from a polymeric film, a foil, paper, paper composite, fibrous webs, laminates thereof or combinations thereof.

9. The gas permeable pouch of claim 1, further comprising an opening device.

10. The gas permeable pouch of claim 9, wherein the opening device comprises a line of weakness along the top margin or a chevron seal.

11. The gas permeable pouch of claim 1, further comprising a closing device.

12. The gas permeable pouch of claim 1, wherein the pouch is self supporting.

13. The gas permeable pouch of claim 1, further comprising a reinforcing member which separates the bottom edge of the front panel from the bottom edge of the back panel.

14. The gas permeable pouch of claim 1, wherein at least a portion of each side margin of the front panel is directly joined to at least a portion of each side margin of the back panel.

15. The gas permeable pouch of claim 1, wherein the bottom edge of the front panel and the bottom edge of the back panel form a bottom perimeter of the pouch and the bottom perimeter has a generally elliptical shape when the bottom panel is fully extended, the bottom panel comprises the gas permeable material, the front panel and the back panel comprise a gas impermeable material and the pouch further comprises an opening device.

16. The gas permeable pouch of claim 15, wherein the gas permeable material comprises a fibrous material, the gas impermeable material comprises a member selected from a polymeric film, a foil, paper, paper composite, fibrous webs, laminates thereof or combinations thereof, and wherein at least a portion of each side margin of the front panel is directly joined to at least a portion of each side margin of the back panel.

17. A sterilization container for an item used in a medical procedure comprising
a front panel and a back panel, each panel comprises a top edge, a bottom edge and two side edges, a top margin at or near the top edge, a bottom margin at or near the bottom edge and two side margins at or near the two side edges, wherein top margin of the front panel is directly or indirectly joined to the top margin of the back panel, each side margin of the front panel is directly or indirectly joined to the one of the side margins of the back panel;
a bottom panel comprising a perimeter having a perimeter margin located at or near the perimeter, wherein the perimeter margin of the bottom panel is directly or indirectly joined to the bottom margin of the front panel and the bottom margin of the back panel and the bottom panel is joined with the bottom margin of the front panel and the bottom margin of the back panel with a line of juncture such that the bottom edge of the front panel and the bottom edge of the back panel are non-linear when the bottom panel is fully expanded, and
wherein the front panel, back panel and bottom panel define a compartment capable of holding one or more articles and at least one the front panel, the back panel or the bottom panel at least partially comprises a gas permeable material; and
an item used in a medical procedure contained within the compartment of the container.

18. The container of claim 17, wherein the item used in a medical procedure comprises a protective garment, a protective covering, a wound covering, a suture, a clamp, a scalpel, a retractor, forceps, scissors, a blade handle, a glove, a needle, a sponge, a syringe, a receptacle, a sealed vessel holding a therapeutic agent or a combination thereof.

19. The container of claim 17, wherein the container is self-supporting and is able to stand upright supporting the weight of the container and the items contained therein.

20. The container of claim 17, further comprising an opening device.

21. The container of claim 20, wherein the opening device comprises a line of weakness along the top margin or a chevron seal.

22. The container of claim 17, further comprising a closing device.

23. The container of claim 18, wherein the bottom edge of the front panel and the bottom edge of the back panel form a bottom perimeter of the pouch and the bottom perimeter has a generally elliptical shape when the bottom panel is fully extended, the bottom panel comprises the gas permeable material, the front panel and the back panel comprise a gas impermeable material and the pouch further comprises an opening device.

24. The container of claim 23, wherein the gas permeable material comprises a fibrous material, the gas impermeable material comprises a member selected from a polymeric film, a foil, paper, paper composite, fibrous webs, laminates thereof or combinations thereof, and wherein at least a portion of each side margin of the front panel is directly joined to at least a portion of each side margin of the back panel.

25. The container of claim 23, wherein the opening device comprises a line of weakness along the top margin or a chevron seal.

26. The container of claim 25, wherein the item used in a medical procedure comprises a protective garment, a protective covering, a wound covering, a suture, a clamp, a scalpel, a retractor, forceps, scissors, a blade handle, a glove, a needle, a sponge, a syringe, a receptacle, a sealed vessel holding a therapeutic agent or a combination thereof.

27. A method of sterilizing an item used in a medical procedure comprising,
providing a sterilization container comprising a front panel and a back panel, each panel comprises a top edge, a bottom edge and two side edges, a top margin at or near the top edge, a bottom margin at or near the bottom edge and two side margins at or near the two side edges, wherein top margin of the front panel is directly or indirectly joined to the top margin of the back panel, each side margin of the front panel is directly or indirectly joined to the one of the side margins of the back panel; a bottom panel comprising a perimeter having a perimeter margin located at or near the perimeter, wherein the perimeter margin of the bottom panel is directly or indirectly joined to the bottom margin of the front panel and the bottom margin of the back panel and the bottom panel is joined with the bottom margin of the front panel and the bottom margin of the back panel with a line of juncture such that the bottom edge of the front panel and the bottom edge of the back panel are non-linear when the bottom panel is fully expanded, wherein the front panel, back panel and bottom panel define a compartment capable of holding one or more articles and at least one the front panel, the back panel or the bottom panel at least partially comprises a gas permeable material; and an item used in a medical procedure contained within the compartment of the sterilization container; and
exposing the sterilization container to a sterilization gas.

28. The method of claim 27, wherein the item used in a medical procedure comprises a protective garment, a protective covering, a wound covering, a suture, a clamp, a scalpel, a retractor, forceps, scissors, a blade handle, a glove, a needle, a sponge, a syringe, a receptacle, a sealed vessel holding a therapeutic agent or a combination thereof.

29. The method of claim 27, wherein the sterilization container is in an upright position with the top edge of the sterilization container being an uppermost portion of the sterilization container, when the sterilization container is exposed to the sterilization gas.

30. The method of claim 29, wherein a plurality of sterilization containers are placed in proximity to one another and at least one of the plurality of sterilization containers is in an upside-down position with the top edge of the sterilization container being the lowermost portion of the sterilization container.

31. The method of claim 27, wherein the sterilization gas comprises ethylene oxide.

32. The method of claim 27, further comprising placing the sterilization container and the item used in a medical procedure is placed into sterilization chamber prior to exposing the container to a sterilization gas.

33. The method of claim 27, further comprising placing the item used in a medical procedure in the compartment of the sterilization container.

34. The method of claim 33, wherein the sterilization container further comprises a closing device and the closing device is closed after the item used in a medical procedure is placed in the compartment of the sterilization container.
